(19) United States
(12) Patent Application Publication
(10) Pub. No.: US 2022/0323498 A1
(43) Pub. Date: Oct. 13, 2022

(54) ASSAY FOR IMMUNE CELL RECOVERY

(71) Applicants: Dean Anthony LEE, Canal Winchester, OH (US); Jena Edwards MOSEMAN, Columbus, OH (US); RESEARCH INSTITUTE AT NATIONWIDE CHILDREN'S HOSPITAL, Columbus, OH (US)

(72) Inventors: Dean Anthony LEE, Canal Winchester, OH (US); Jena Edwards MOSEMAN, Columbus, OH (US)

(21) Appl. No.: 17/641,473

(22) PCT Filed: Sep. 10, 2020
(86) PCT No.: PCT/US2020/050112

§ 371 Date: Mar. 9, 2022

Related U.S. Application Data
(60) Provisional application No. 62/898,170, filed on Sep. 10, 2019.
(60) Provisional application No. 62/904,843, filed on Sep. 24, 2019.

(51) Int. Cl. A61K 35/17; G01N 33/50; G01N 33/68; A61K 38/17

(52) U.S. Cl. CPC A61K 35/17; G01N 33/5047; G01N 33/6872; A61K 38/1774; A61K 38/1793; A61K 38/177; G01N 2333/96494

(57) ABSTRACT

Disclosed are compositions and methods for measuring the likelihood of recovery of a recently thawed immune cell. Methods include assaying the level of an ADAM-17-cleaved surface receptor expressed on the immune cells, wherein the level of the surface receptor directly correlates with the likelihood of immune cell recovery (i.e., the greater the increase of the expression level an ADAM-17-cleaved surface receptor relative to a control, the greater the likelihood of recovery). The method can be used to determine if immune cells have sufficient viability to be used in immunotherapy before use.

I. This application claims the benefit of U.S. Provisional Application No. 62/904,843, filed on Sep. 24, 2019 and U.S. Provisional Application No. 62/898,170, filed on Sep. 10, 20219, applications which are incorporated herein by reference in their entirety.

### II. BACKGROUND

Immune cell cryopreservation has been a difficult problem to overcome for immunotherapy. Variability in the viability of different lots or different donors of cryopreserved immune cells is also a significant problem for cell-based immunotherapy. In addition, common assays of viability performed shortly after thawing may have poor predictive value of functional recovery. For example, T cells show a greater than 90% variability after thaw between groups. Immune cells such as natural killer (NK) cells that are cryopreserved often have poor recovery and function post-thaw. NK cells immediately after thaw have 80-90% viability, but they continue to lose viability over the next 24 hours, with final viable recovery often as low as 20%. Therefore, the viability immediately upon thaw is not a good indication of whether or not the cells will survive or be highly functional.

Interest in NK cell-based immunotherapy has resurged since new protocols for the purification and expansion of large numbers of clinical-grade cells have become available. However, because such cells are cryopreserved before use, there remains a need for a reliable method to determine the viability of NK cells after cryopreservation to assure that cells administered are dosed according to numbers of viable, effective cells.

### III. SUMMARY

Disclosed are methods related to measuring cell recovery following a cell membrane damaging event based on the surrogate measurement of cell-surface surface receptors (such as, for example, CD16) that are cleaved by metalloproteases (such as, for example, ADAM17) that were activated by calcium entering the cell during the cell membrane damaging event.

The inventors have discovered that there is loss of CD16 receptor due to cleavage by ADAM17 on the day of thaw, but 24 hours after that the surface level of CD16 is recovered on viable cells. Without being bound by theory, evidence is consistent with the belief that metalloproteases, such as ADAM17, have detrimental effects on NK cell function via the shedding of CD16. CD16 is a crucial protein that is expressed on NK cells, as it is responsible for antibody-dependent and antibody-independent cytotoxicity of NK cells. The inventors have identified a technology that can predict how well NK cells will recover by analyzing the percentage of CD16 present in the live cell population on the day of thawing of cryopreserved samples. This could benefit immune cell therapy by having an immediate marker of recovery on the day of thaw, ensuring patients will only receive functional, viable NK cell products for infusion.

In one aspect, disclosed herein are methods of measuring the likelihood of recovery of an immune cell (such as, for example, a T cell, natural killer (NK) cell, macrophages, dendritic cells, natural killer T (NKT) cells, innate lymphoid cells (ILCs), B cells, γδT cells, neutrophils, chimeric antigen receptor (CAR) T cell, and/or CAR NK cell) following a cell membrane damaging event (including, but not limited to a freeze thaw cycle, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure), comprising assaying the level of an ADAM-17-cleaved surface receptor (such as, for example CD16, CD62L, or IL-15 receptor (IL-15R)) expressed on the immune cells (including, but not limited to assaying via flow cytometry), wherein an increase in the level of the surface receptor (relative to a control immune cell or relative to a mixed population of cell membrane damaged immune cells) directly correlates with the likelihood of immune cell recovery.

Also disclosed herein are methods of measuring the likelihood of recovery of an immune cell after a cell membrane damaging event of any preceding aspect, wherein the level of ADAM-17-cleaved surface receptor expression is assayed within 0 to 24 hours or 12 to 24 hours after the immune cell membrane damaging event.

In one aspect, disclosed herein are methods of measuring the likelihood of recovery of an immune cell after a cell membrane damaging event of any preceding aspect, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

Also disclosed herein are methods of administering an immunotherapy (such as, for example, anticancer treatment) to a subject in need thereof, comprising a) obtaining one or more immune cells (such as, for example, a T cell, natural killer (NK) cell, macrophages, dendritic cells, natural killer T (NKT) cells, innate lymphoid cells (ILCs), B cells, γδT cells, neutrophils, chimeric antigen receptor (CAR) T cell, and/or CAR NK cell) previously subjected following a cell membrane damaging event (including, but not limited to a freeze thaw cycle, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure); b) assaying the expression level of an ADAM-17-cleaved surface receptor (such as, for example CD16, CD62L, or IL-15 receptor (IL-15R)); and c) administering to the subject a therapeutically effective amount of immune cells that express an increased level of ADAM-17-cleaved surface receptor relative to a control immune cell or relative to a mixed population of cell membrane damaged immune cells.

In one aspect, disclosed herein are methods of administering an immunotherapy of any preceding aspect, wherein the level of ADAM-17-cleaved surface receptor expression is assayed within 0 to 24 hours or 12 to 24 hours after the immune cell membrane damaging event.

Also disclosed herein are methods of administering an immunotherapy of any preceding aspect, wherein the level of ADAM-17-cleaved surface receptor expression is assayed using flow cytometry.

In one aspect, disclosed herein are methods of administering an immunotherapy of any preceding aspect, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

Also disclosed herein is use in an immunotherapy of a therapeutically effective amount of immune cells previously subjected following a cell membrane damaging event, wherein the cells express an increased level of ADAM-17-cleaved surface receptor relative to a control immune cell or relative to a mixed population of cell membrane damaged immune cells. In one aspect, the immunotherapy may be, for example, an anticancer treatment.

Also disclosed herein is an immunotherapeutic composition comprising a therapeutically effective amount of immune cells previously subjected following a cell membrane damaging event, and expressing an increased level of ADAM-17-cleaved surface receptor relative to a control immune cell or relative to a mixed population of cell membrane damaged immune cells. The immunotherapeutic composition may further comprise a pharmaceutically acceptable carrier.

It should be understood that the various aspects of any of the methods, uses and immunotherapeutic compositions described herein, encompass methods, uses and compositions in which the cell membrane damaging event may comprise any one of a freeze thaw cycle, gene editing, electroporation, magnetofection, detergent permeabilization, strotolysin O (SLO) exposure, physical morphology change (cell squeezing), and/or ethanol exposure. In any of the methods, uses and compositions described herein, the level of ADAM-17-cleaved surface receptor expression may be determined within 0 to 24 hours, or within 12-24 hours after the cell membrane damaging event. In any of the methods, uses and compositions described herein, the ADAM-17-cleaved surface receptor expressed on the immune cells may comprise CD16, CD62L, or IL-15 receptor (IL-15R). In any of the methods, uses and compositions described herein, the immune cells may comprise a T cell, Natural Killer (NK) cell, chimeric antigen receptor (CAR) T cell, macrophage, dendritic cells, natural killer T (NKT) cells, innate lymphoid cell (ILC), B cell, γδT cell, neutrophil, and/or a CAR NK cell. When the immune cells comprise Natural Killer (NK) cells and/or CAR NK cells, the ADAM-17-cleaved surface receptor may comprise CD16, and/or the cells may comprise expanded NK or CAR NK cells. When the immune cells comprise T cells and/or CAR T cells, the ADAM-17-cleaved surface receptor may comprise CD62L or IL-15R. In any of the methods, uses and compositions described herein, the level of ADAM-17-cleaved surface receptor expression may be assayed using flow cytometry, and/or may be expressed as a ratio of the level of surface receptor cleaved by ADAM17 expressed on the cell membrane damaged immune cell compared with the normal level of ADAM-17-cleaved surface receptor expressed on the immune cells.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 provides graphs showing the gating strategy for measuring the percentage of live lymphocytes expressing CD16.

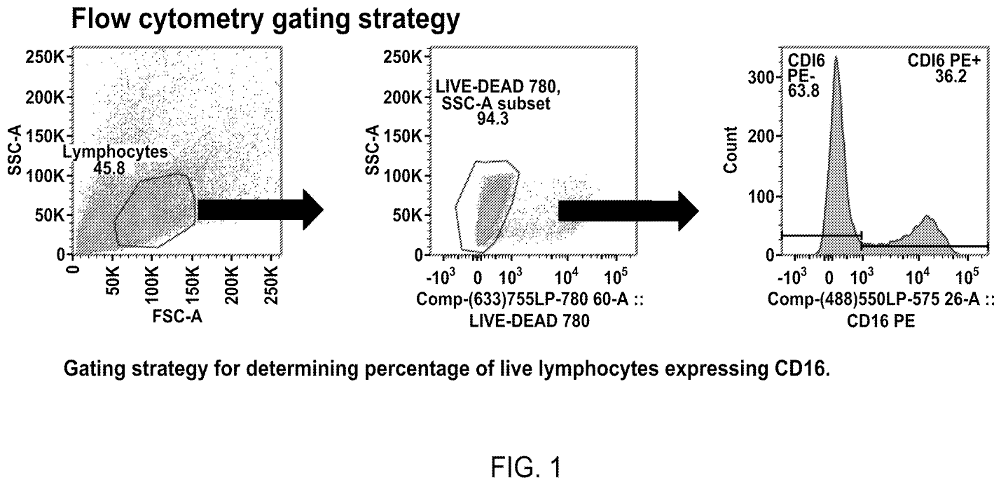

FIG. 2 provides graphs showing that CD16 expression on NK cells is lost immediately after thawing, but is recovered after overnight resting in complete media and IL-2.

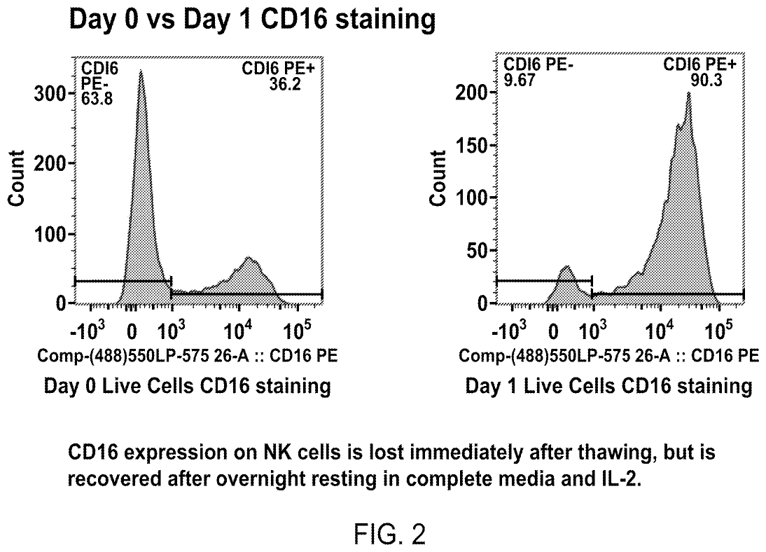

FIG. 3 provides a graph showing there is a positive correlation between CD16 expression on Day 0 and the percentage of NK cells that recover after overnight resting in complete media and IL-2 is consistent across 7 different NK cell donors.

FIG. 4 provides a graph showing a positive correlation between CD16 expression on Day 0 and the percentage of NK cells that recover after overnight resting in complete media and IL-2 is consistent across 9 different types of cryopreservation media.

FIG. 5 provides a graph showing a positive correlation between CD16 expression on Day 0 and the percentage of NK cells that recover after overnight resting in complete media and IL-2 across 5 different experiments that is consistent across time and experimental conditions.

FIG. 6 provides figures showing that the loss of CD16 is regulated by ADAM17, wherein NK cells in which ADAM17 is deleted have enhanced recovery of CD16 expression.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

26. “Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

29. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

35. “Biocompatible” generally refers to a material and any metabolites or degradation products thereof that are generally non-toxic to the recipient and do not cause significant adverse effects to the subject.

36. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

38. “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

40. “Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

41. “Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

42. “Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

43. “Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

44. “Immune cell(s)” refers to any immune cell such as T cells, natural killer (NK) cells, macrophages dendritic cells, γδT cells, natural killer T (NKT) cells, innate lymphoid cells (ILCs), B cells, neutrophils, chimeric antigen receptor (CAR) T cell, and/or CAR NK cell), or any combination thereof.

It should be understood that “ADAM-17-cleaved surface receptor” encompasses receptors that may also be subject to shedding or loss mediated by other proteases.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Measuring the Likelihood of Cell Recovery

The present invention provides a method of measuring the likelihood of recovery of an immune cell after a cell membrane damaging event (such as, for example, a freeze thaw cycle such those that occur through cryopreservation, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure). The method includes measuring the level of an ADAM-17-cleaved surface receptor expressed on the immune cells as a surrogate for damage-induced ADAM-17 activity, wherein an increased level of the surface receptor (i.e., levels of the ADAM-17-cleaved surface receptor are maintained or have less loss of the ADAM-17-cleaved surface receptor) relative to an undamaged cell) directly correlates with less damage and a greater likelihood of immune cell recovery. That is, the less the loss of the ADAM-17-cleaved surface receptor the greater likelihood of immune cell recovery. In one aspect, the present invention provides a method of measuring the likelihood of recovery of an immune cell after a cell membrane damaging event (such as, for example, a freeze thaw cycle such those that occur through cryopreservation, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure), comprising assaying the level of an ADAM-17-cleaved surface receptor (such as, for example CD16, CD62L, or IL-15 receptor (IL-15R)) expressed on the immune cells, wherein an increased level of the surface receptor directly correlates with the likelihood of immune cell recovery. That is, the greater the surface expression of the ADAM-17-cleaved surface receptor on the immune cell (i.e., the smaller the loss of the surface receptor due to ADAM-17 cleavage and the more closely the surface receptor levels are to an undamaged control), the more likely the immune cell will recover. Accordingly, disclosed herein are methods of measuring the likelihood of recovery of an immune cell after a cell membrane damaging event, comprising assaying the level of an ADAM-17-cleaved surface receptor expressed on the immune cells, wherein an increase in the level of the surface receptor directly correlates with the likelihood of immune cell recovery.

The methods disclosed herein allow for the ability to assess the likelihood of recovery from a cell membrane damaging event. Immune cell recovery refers to a restoration of function by the immune cell. Live (viable) cells are recovered cells, but some live cells exhibit a degree of loss of normal function. Cell viability can be determined, for example, but not limited to, using viable cell counts were performed using trypan blue exclusion. Alternatively, any of many methods known in the art for determining cell viability can be used. Preferably, restored cells include all, or at least a significant portion, of the typical activities seen for the particular type of immune cell. Cell function can be evaluated using a variety of assays known to those skilled in the art.

It is understood and herein contemplated that a “cell membrane damaging event” can refer to any event or manipulation of the cell that results in distortion or permeabilization (such as, for example, permanent, reversible, or transient permeabilization) of the cell membrane. Such events can include, but are not limited to a freeze thaw cycle (such as that which occurs as a result of cryopreservation), gene editing, electroporation, magnetofection, detergent permeabilization (such as, for example, permeabilization with saponin and/or digitonin), strotolysin O (SLO) exposure, physical morphology change (cell squeezing), and/or ethanol (including, but not limited solutions comprising 30% or less ethanol) exposure.

For example, cryopreservation of immune cells can be carried out using a freezing and thawing process (and equipment) in which the cells are frozen at a rate of about 1° C. per minute until they reach −80° C. or liquid nitrogen temperatures of about −200° C., where they may be stored indefinitely, and after which must be thawed very rapidly. Immune cells may be frozen after they have been isolated, or they may be frozen in a blood sample. Immune cells can be cryopreserved using a variety of different apparatus, such as a Mr. Frosty®, which are polycarbonate freezing containers provided by Nalgene®, shock freezing, freezing in a Styrofoam insulator, or using a controlled rate freezer. A storage temperature of at or about −80° C. is preferred. Often, dimethyl sulfoxide (DMSO) or another cryopreservative (e.g., glycerol) is also used in order to help protect the cells. Typically, about 5% to about 15% DMSO and/or sugar-based cryoprotectants such as glycerol, ethylene glycol, dextran, hydroxyethyl starch, or trehalose are used. Once frozen, an immune cell will have a relatively long shelf life, and can then be thawed when needed. When the immune cells are needed, they can be thawed. Thawing refers to the process of defrosting the cells, i.e., raising the temperature above the freezing point. The cells may further be warmed to a normal cell culture temperature, such as about 37° C. The present invention provides a method of measuring the likelihood of recovery of a recently thawed immune cell.

Immune cells may be thawed using a variety of methods known to those skilled in the art. For example, immune cells can be thawed using a water bath, or a controlled heat-transfer device. A water bath, as used herein, is a laboratory water bath. Water baths are available in a variety of volumes, and include a temperature control to heat the water in the bath to a desired temperature. Water baths often include various means of providing an even temperature within the water bath, including means for moving the water, such as circulation and shaking. For example, a variety of suitable laboratory water baths can be obtained from Thermo Scientific, Benchmark Scientific, or Sheldon Manufacturing, Inc. Preferably, the water bath includes water at a temperature of 37° C., which will raise the temperature of the immune cells to body temperature after thawing is complete. A controlled heat transfer device accomplishes the same process without using water as the medium of heat transfer, such as the VIAThaw device from Asymptote, Ltd. (part of GE Healthcare Life Sciences, now Cytiva). Generally, frozen immune cells should be thawed immediately after removal from frozen storage. Immune cells should then be thawed quickly (i.e., within a few minutes) by immersing the container holding the cells in the water bath until there is just a small bit of ice remaining in the container with the cells. The cells are then transferred to a pre-warmed growth medium appropriate for the cells (e.g., RPMI-1640 medium). Additional steps, such a centrifuging and re-suspending the immune cells in new growth medium, can also be carried out.

As disclosed herein, the likelihood of recovery of an immune cell from cell membrane damage directly correlates with ADAM-17 activation, which in turn can be evaluated by assaying the level of an ADAM17-cleaved surface receptor expressed on the immune cells. ADAM metallopeptidase domain 17 (ADAM17), also called TACE (tumor necrosis factor-α-converting enzyme), is a 70-kD an enzyme that belongs to the ADAM protein family of disintegrins and metalloproteases. ADAM17 is understood to be involved in the release of a diverse variety of membrane-anchored cytokines, cell adhesion molecules, receptors, ligands, and enzymes in a process known as “shedding.” ADAM17 can cut off the part of a transmembrane receptor which has already bound an agonist (e.g., CD16, CD62L, and/or IL-15R), allowing the agonist to go and stimulate a receptor on another cell. The ADAM-17 cleaved surface receptors expressed on the immune cells can vary depending on the type of immune cell. For example, in some embodiments the immune cell is a Natural Killer (NK) cell, and the ADAM-17-cleaved surface receptor is CD16. In other aspects, the immune cell is a T cell and the ADAM-17-cleaved surface receptor is CD62L or IL-15R.

Immune cells, as defined herein, are any cells of the immune system that produce cytokines (i.e., cytokine-producing immune cells). Examples of cytokine-producing immune cells include lymphocytes, neutrophils, macrophages, and natural killer cells. Lymphocytes include B cells and T cells and NK cells. In some embodiments, the immune cell is a a T cell, natural killer (NK) cell, macrophage, dendritic cell, natural killer T (NKT) cell, innate lymphoid cell (ILC), B cell, γδT cell, neutrophil, chimeric antigen receptor (CAR) T cell, and/or CAR NK cell). The immune cells can be obtained from cell culture, or can be obtained from a subject.

In some embodiments, the immune cell is a T-cell. T-cells play a central role in cell-mediated immunity, and can be distinguished from other lymphocytes, such as B cells and natural killer cells, by the presence of a T-cell receptor on the cell surface. Examples of T-cells include T helper cells (TH cells), cytotoxic T cells (TC cells), effector T cells, memory T cells, regulatory or “suppressor” T cells, γδT cells, and Natural killer T cells (NKT cells, which are distinct from NK cells and recognize a glycolipid antigen rather than peptides presented by the MHC molecule). Different types of T-cells differ from each other in their pattern of cytokine production.

In some embodiments, the immune cell is an NK cell. Natural Killer Cells are a type of cytotoxic lymphocyte of the immune system. NK cells provide rapid responses to virally infected cells and respond to transformed cells. Typically, immune cells detect peptides from pathogens presented by Major Histocompatibility Complex (MHC) molecules on the surface of infected cells, triggering cytokine release, causing lysis or apoptosis. NK cells are unique, since they have the ability to recognize stressed cells regardless of whether peptides from pathogens are present on MHC molecules. They were named “natural killers” because of the initial notion that they do not require prior activation in order to kill target. NK cells are large granular lymphocytes (LGL) and are known to differentiate and mature in the bone marrow from where they then enter into the circulation.

In some embodiments, the immune cells are immunotherapeutic immune cells. Immunotherapeutic immune cells are those that are useful for treatment of diseases such as cancer. Immunotherapeutic immune cells include tumor infiltrating lymphocytes (TILs), T cells, and/or NK cells, which have been described for use in the treatment of cancer. Immunotherapeutic immune cells also cells that have been manipulated to comprise chimeric antigen receptors (CARs) including, but not limited to CAR T cells and CAR NK cells, which have also been used for the treatment of cancer.

Because it is helpful to be able to administer large numbers of immune cells during immunotherapy, in some embodiments the immune cells are expanded immune cells. Expanded immune cells are those that are grown ex-vivo in order to grow a large number of immune cells. Expanded immune cells can be expanded from immune cells of the same time, or they can be expanded from other cell types. For example, NK cells can be expanded from peripheral blood mononuclear cells or hematopoietic stem cells. In some embodiments, the expanded immune cells are autologous cells that can be easily administered to a subject without provoking an immune response. However, in some embodiments, the expanded immune cells are allogeneic immune cells, in which their inherent alloreactivity can be a benefit. In further embodiments, the expanded immune cells are genetically engineered to include chimeric antigen receptors to help the immune cells target diseased tissue. Preparation of expanded immune cells includes activating and expanding the immune cells. A number of cytokines (IL-2, IL-12, IL-15, IL-18, IL-21, type I IFNs, and TGF-β) have been shown to be useful for activating and expanding NK cells ex vivo. For example, in some embodiments, the NK cells being evaluated are IL-21 expanded NK cells. IL-21 expanded NK cells includes NK cells stimulated by soluble IL-21, feeder cells comprised with membrane bound IL-21 (mbIL-21), plasma membrane particles containing mbIL-21, exosomes containing mbIL-21, and solid supports with mbIl-21.

Measuring the likelihood of recovery of the immune cell following a cell membrane damaging event (such as, for example, a freeze thaw cycle such those that occur through cryopreservation, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure) can occur any time after the cell membrane damaging event. A recently damaged immune cell is one which was damaged within the last 48 hours. In some embodiments, a recently damaged immune cell is one which was damaged within 0 to 24 hours, while in other embodiments, a recently damaged immune cell is one which was damaged within 0 to 12 hours. In further embodiments, a recently damaged immune cell is one which was damaged within 12 to 24 hours. For example, an immune cell can be assayed within 5, 10, 15, 20, 25, 30, 35, 40, 45, 50, 55, 60, 75, 90, 105, 120, 150, 180 minutes, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours after the immune cell membrane damaging event.

In the method of measuring the likelihood of recovery of an immune cell following a cell membrane damaging event (including, but not limited to a freeze thaw cycle, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure), an increased level of the ADAM-17-cleaved surface receptor directly correlates with the likelihood of immune cell recovery. That is, the greater the increase in ADAM-17-cleaved surface receptor expression (i.e., less surface receptor has been cleaved by ADAM17 and the closer the expression levels are to undamaged controls), the greater the likelihood of immune cell recovery. Similarly, the greater the reduction in ADAM-17-cleaved surface receptor expression the less likely a cell is to recover. An increased level of ADAM-17-cleaved surface receptor can be determined by comparison with control values. The control values can be previously identified control values, or they can be control values that are measured at the same time as the ADAM-17-cleaved surface receptor levels are determined. Controls can be positive controls such as undamaged cells where comparisons can be made relative to the amount of loss with cells most likely to recover being cells that have less loss of ADAM-17-cleaved surface receptors and greater loss of ADAM-17-cleaved surface receptors are less likely to recover. Alternatively, controls can be negative controls that are cell surface damaged or undamaged that have low or no ADAM-17-cleaved surface receptor expression and cells that are likely to recover have increased ADAM-17-cleaved surface receptor expression relative to that control. As the ability to recover is a direct correlation, with more recover being seen with increases in The increase or decrease can also be relative to other immune cells in the population with the cells expressing increased ADAM-17-cleaved surface receptor more likely to recover than cells from the same sample with less ADAM-17-cleaved surface receptor.

In some embodiments, the level of ADAM-17-cleaved surface receptor expressed on the immune cells is expressed as a ratio of the level of surface receptor cleaved by ADAM17 expressed on the damaged immune cell compared with the normal level of ADAM-17-cleaved surface receptor expressed on the immune cells. Thus, in one aspect, disclosed herein are methods of measuring the likelihood of recovery of an immune cell after a cell membrane damaging event, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

The amount of ADAM-17-cleaved surface receptor can be assayed using any method capable of detecting the amount of protein on a cell surface. For example, the amount of ADAM-17-cleaved surface receptor can be detected using immunoassay or cell sorting methods. Immunoassays come in many different formats and variations. Immunoassays may be run in multiple steps with reagents being added and washed away or separated at different points in the assay. Immunoassays include heterogeneous immunoassays, which include multiple steps, and homogenous immunoassays, which involve simply mixing the reagents and sample and making a physical measurement. Types of immunoassays include competitive, homogenous immunoassays, competitive heterogenous immunoassays, one-site non-competitive immunoassays, and two-site noncompetitive immunoassays. Immunoassays also include Enzyme-linked immunosorbent assays (ELISA), lateral flow immunoassays, enzyme-linked immunosorbent spot (ELIspot) assays, antibody array assays and bead-based assays, magnetic immunoassays, western blots, and radioimmunoassays.

In some embodiments, the level of ADAM-17-cleaved surface receptor expression is assayed using flow cytometry. Flow cytometry is a cell-sorting method in which cells are labeled with fluorescent markers, and then run through the flow cytometer where light scattering is used to characterize the cells. Similarly, mass cytometry (CyTOF) may be used for assays.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

The sample is run for example in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Omstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, α-glycerophosphate dehydrogenase, triose phosphate isomerase, glucose-6-phosphate dehydrogenase, glucoamylase and acetylcholinesterase.

Variations of ELISA techniques are known to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunocomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunocomplex (antigen/antibody) formation. Detection of the immunocomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, α-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

95. “Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialized chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include color coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatized with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumor extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilized on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### C. Method of Immunotherapy

The present invention also provides a method of immunotherapy comprising administering a therapeutically effective amount of immune cells to a subject in need thereof, wherein the immune cells were recently damaged and were determined to be viable before administration using the method of likelihood of recovery described herein. Thus, in one aspect, disclosed herein are methods of administering an immunotherapy (such as, for example, anticancer treatment) to a subject in need thereof, comprising a) obtaining one or more immune cells (such as, for example, a T cell, natural killer (NK) cell, macrophage, dendritic cell, neutrophil, γδT cell, natural killer T (NKT) cell, innate lymphoid cell (ILCs), B cell, chimeric antigen receptor (CAR) T cell, and/or CAR NK cell) previously subjected following a cell membrane damaging event (including, but not limited to a freeze thaw cycle, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure); b) assaying the expression level of an ADAM-17-cleaved surface receptor (such as, for example CD16, CD62L, or IL-15 receptor (IL-15R)); and c) administering to the subject a therapeutically effective amount of immune cells that express an increased level of ADAM-17-cleaved surface receptor relative to a control immune cell or relative to a mixed population of cell membrane damaged immune cells.

As noted above, the method of immunotherapy includes administering a therapeutically effective amount of immune cells to a subject in need thereof, wherein the immune cells subjected following a cell membrane damaging event (including, but not limited to a freeze thaw cycle, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure) and were determined to be viable and recovered before administration by determining that they expressed an increased level of ADAM-17-cleaved surface receptor. Recovered cells are more likely to provide effective immunotherapy and are more likely to survive the administration process (thus increasing the take of transferred cells), and as a result it is useful to be able to evaluate the recovery of immune cells before they are administered for immunotherapy. In one aspect, it is understood and herein contemplated that by measuring recovery of the immune cells (by assaying the expression level of an ADAM-17-cleaved surface receptor) the practicing physician can select for transfer cells that have recovered (i.e., express an increased level of ADAM-17-cleaved surface receptor) thus increasing the percentage of cells likely to survive transfer to the subject and be immunotherapeutic to the subject.

Immunotherapy, as used herein, refers to cell-based immunotherapy in which immune cells such as lymphocytes (including, but not limited to tumor infiltrating lymphocytes (TILs)), macrophages, dendritic cells, natural killer (NK) cells, natural killer T (NKT) cells, innate lymphoid cells (ILCs), B cells, γδT cells, neutrophils, cytotoxic T lymphocytes, chimeric antigen receptor (CAR) T cells, and/or CAR NK cells are administered to a subject to achieve a therapeutic effect. Cell-based immunotherapy is most frequently used as an anticancer treatment for a subject who has been diagnosed as having cancer. Cell-based immunotherapy includes adoptive cell transfer, in which immune cells are extracted from the patient or another individual and then administered to improve immune functionality. For example, in autologous cancer immunotherapy, T-cells or NK cells are extracted from a patient, optionally genetically modified, and cultured in vitro, and then returned to the same patient. Alternately, allogeneic cell-based immunotherapy involves cells isolated and expanded from a donor separate from the patient receiving the immune cells. In some embodiments, method of immune therapy includes the use of T-cells or Natural Killer (NK) cells that have experienced a cell membrane damaging event (including, but not limited to a freeze thaw cycle, gene editing, electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol). In further embodiments, the method of immune therapy includes the use of NK cells, such as expanded NK cells.

Because of the delay between when the cells are obtained, and when the cells are needed for immunotherapy, it is common to freeze the cells so that they can be stored until needed. Methods of freezing and thawing immune cells are described herein, and are known to those skilled in the art. In some embodiments, the immune cells were thawed using a water bath. In other aspects, the cells may be manipulated to provide more effective treatment such as by inserting a therapeutic vector or peptide, gene editing, or the construction of chimeric antigen receptors. These manipulations often involve permeabilization of the cell membrane via electroporation, magnetofection, detergent permeabilization (such as, for example, saponin and/or digitonin), strotolysin O (SLO) exposure, physical morphology change (cell squeezing), and/or ethanol (including, but not limited solutions comprising 30% or less ethanol) exposure. It is understood and herein contemplated that these manipulations or cryopreservation efforts (i.e., free thaw cycles) result in cell membrane damage.

Measuring the likelihood of recovery of the immune cell following a cell membrane damaging event (such as, for example, a freeze thaw cycle such those that occur through cryopreservation, gene editing, electroporation, magnetofection, cell squeezing, detergent permeabilization (such as, for example, saponin and/or digitonin), strotolysin O (SLO) exposure, physical morphology change (cell squeezing), and/or ethanol (including, but not limited solutions comprising 30% or less ethanol) exposure) for use in the disclosed immunotherapy methods can occur any time after the cell membrane damaging event. A recently damaged immune cell is one which was damaged within the last 48 hours. In some embodiments, a recently damaged immune cell is one which was damaged within 0 to 24 hours, while in other embodiments, a recently damaged immune cell is one which was damaged within 0 to 12 hours. In further embodiments, a recently damaged immune cell is one which was damaged within 12 to 24 hours. For example, an immune cell can be assayed within 5, 10, 15, 20, 25, 30, 35, 40, 45, 50, 55, 60, 75, 90, 105, 120, 150, 180 minutes, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours after the immune cell membrane damaging event.

Measuring the likelihood of recovery of the immune cell following a cell membrane damaging event can occur using any method capable of detecting the amount of protein on a cell surface. For example, the amount of ADAM-17-cleaved surface receptor can be detected using immunoassay or cell sorting methods. Immunoassays come in many different formats and variations. Immunoassays may be run in multiple steps with reagents being added and washed away or separated at different points in the assay. Immunoassays include heterogeneous immunoassays, which include multiple steps, and homogenous immunoassays, which involve simply mixing the reagents and sample and making a physical measurement. Types of immunoassays include competitive, homogenous immunoassays, competitive heterogenous immunoassays, one-site non-competitive immunoassays, and two-site noncompetitive immunoassays. Immunoassays also include Enzyme-linked immunosorbent assays (ELISA), lateral flow immunoassays, enzyme-linked immunosorbent spot (ELIspot) assays, antibody array assays and bead-based assays, magnetic immunoassays, and radioimmunoassays.

In the method of immunotherapy using immune cells previously subject following a cell membrane damaging event, an increased level of the ADAM-17-cleaved surface receptor directly correlates with the likelihood of immune cell recovery. That is, the greater the increase in ADAM-17-cleaved surface receptor expression, the greater the likelihood of immune cell recovery. In some embodiments, disclosed herein are methods of administering an immunotherapy, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, Pa.

Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

In one aspect, disclosed herein are methods of administering an immunotherapy, wherein the immunotherapy is an anticancer treatment for a subject who has been diagnosed as having a cancer. The disclosed immunotherapeutic methods can be used to treat, inhibit, reduce, decrease, ameliorate, and/or prevent any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Thus, in one aspect disclosed herein are methods of treating, inhibiting, decreasing, reducing, ameliorating, and/or preventing a cancer and/or metastasis in a subject comprising a) obtaining one or more immune cells previously subjected following a cell membrane damaging event; b) assaying the expression level of an ADAM-17-cleaved surface receptor; and c) administering to the subject a therapeutically effective amount of immune cells that express an increased level of ADAM-17-cleaved surface receptor relative to a control immune cell.

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Percentage CD16 and Overall Recovery

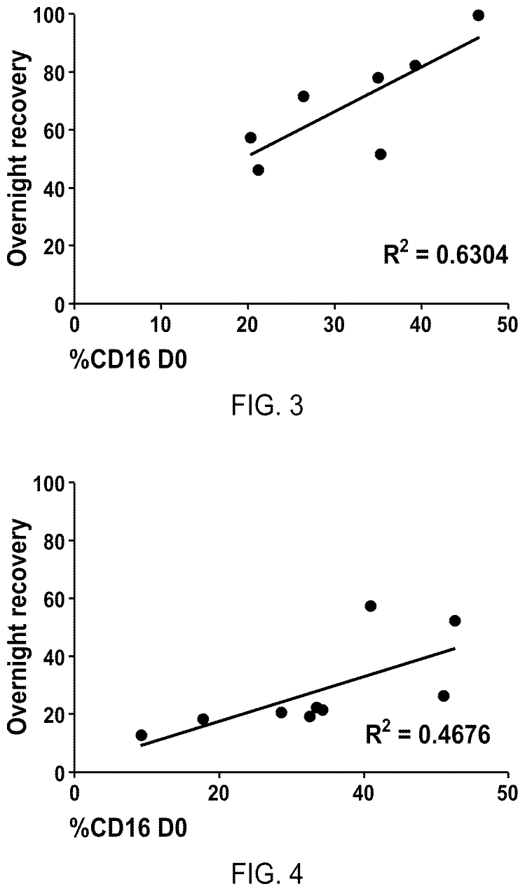

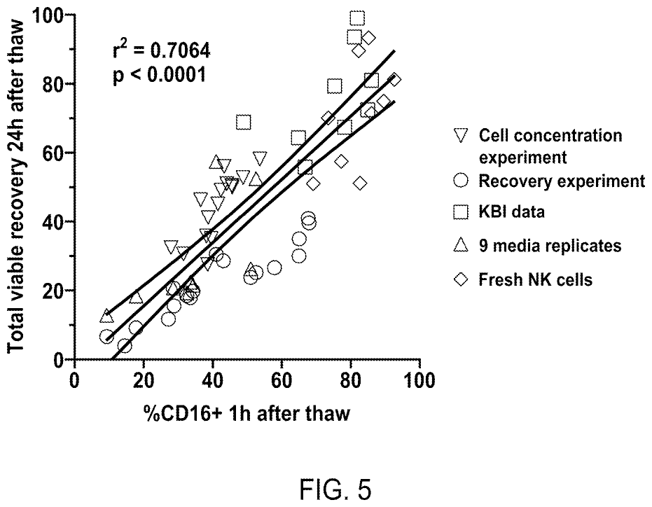

Natural Killer cells were expanded ex-vivo for 14 days using K562 mbIL21.41bbL feeder cells. NK cells were frozen in nine different cryopreservation medias on Day 14, and frozen at a rate of 1° C./minute using a Mr. Frosty Device. NK cells were thawed in a 37° C. water bath using standard protocols, counted, and assessed for flow cytometric analysis immediately after thaw. All cells were rested overnight in a T25 flask containing the same number of viable cells in each condition. NK cells were counted on Day 1 post-thaw. Total recovery from Day 0 and Day 1 was calculated, and is depicted in FIGS. 3 and 4.

The methods used to generate these results are described here in greater detail. On day 14 of cell expansion, a viable cell count was completed using trypan blue cell exclusion. The cells were then frozen using different cryopreservation media, as described above. On day 0 of the thaw, cells were thawed using a 37° C. water bath with slight agitation until only one small piece of ice remained in the vial. Media was then added to the thawed cell suspension, and the thawed cells in media were spun at 400×g for 5 minutes in order to separate out the cells. The cells were then resuspended in complete media, and viable cell counts were performed using trypan blue exclusion. An aliquot of NK cells were then stained for CD16 and a viability dye using a common flow cytometry staining protocol. Using the initial cell suspension, a specified number of viable cells were then allowed to rest overnight in complete media containing IL-2.

On day 1 after the thaw, viable cell counts were performed using trypan blue exclusion. The percent recovery of cells was calculated from overnight rest.

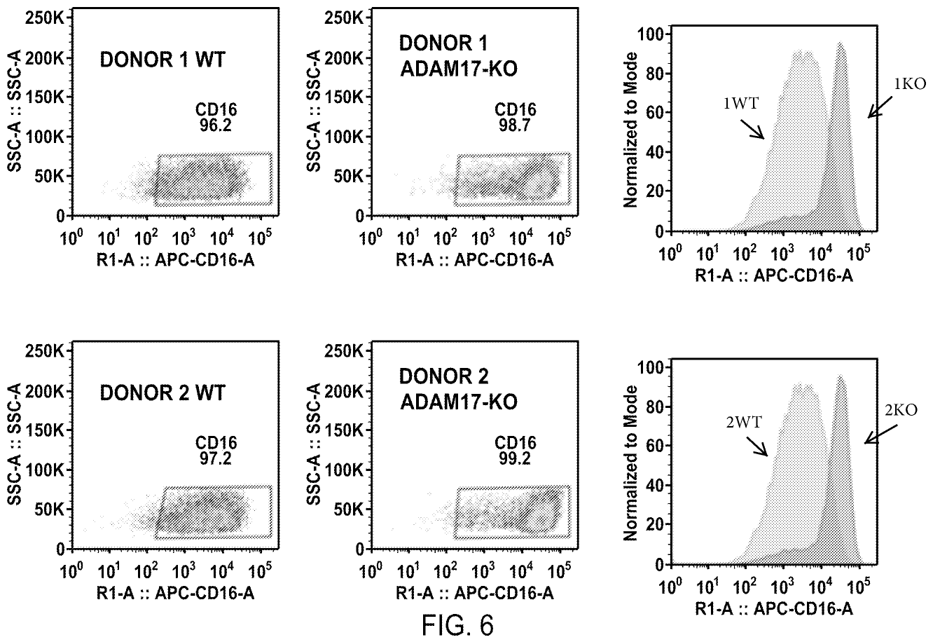

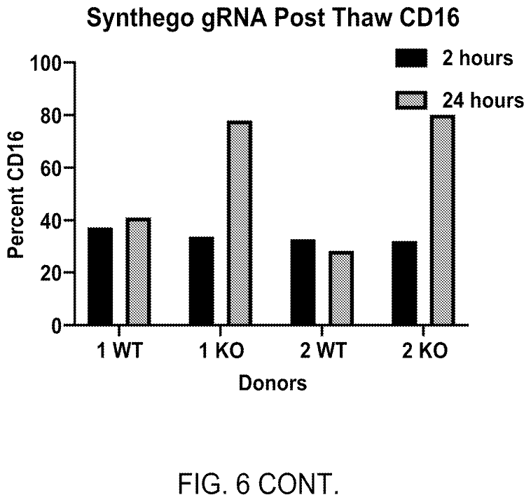

As shown in FIG. 5, NK cells were thawed, washed of cryopreservation media, resuspended in culture media, and rested for 24 h recovery. CD16 expression on NK cells was determined by flow cytometry within 60 minutes after thawing. The loss of CD16 is regulated by ADAM17, wherein NK cells in which ADAM17 is deleted have enhanced recovery of CD16 expression (FIG. 6).

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

### E. References

Becker et al., Cancer Immunol. Immunother 65, 477-484 (2016).
Bethune et al., Curr Opin Biotechnol., 48:142-152 (2017).
Fang et al., Semin Immunol., 31:37-54 (2017).
Koepsell et al., Transfusion, 53(2):404-10 (2013).
Luo et al., Cryobiology, 79:65-70 (2017).
Mandelboim et al., Proceedings of the National Academy of Sciences of the United States of
America, 96(10):5640-5644 (1999).
Rezvani et al., Front Immunol., 6, 578 (2015).
Worrell et al., Diabetes Metab Res Rev, 27(8):737-45 (2011).
Xin et al., Proc Natl Acad Sci USA. 114(4):740-745 (2017).

What is claimed is:

1. A method of measuring the likelihood of recovery of an immune cell after a cell membrane damaging event, comprising assaying the level of an ADAM-17-cleaved surface receptor expressed on the immune cells, wherein an increase in the level of the surface receptor directly correlates with the likelihood of immune cell recovery.

2. The method of measuring the likelihood of recovery of an immune cell after a cell membrane damaging event of claim 1, wherein the membrane damaging event comprises a freeze thaw cycle, gene editing, electroporation, magnetofection, detergent permeabilization, strotolysin O (SLO) exposure, physical morphology change (cell squeezing), and/or ethanol exposure.

3. The method of measuring the likelihood of recovery of an immune cell after a cell membrane damaging event of claim 2, wherein the membrane damaging event comprises a freeze thaw cycle.

4. The method of measuring the likelihood of recovery of an immune cell after a cell membrane damaging event of claim 3, wherein immune cells were thawed using a water bath.

5. The method of measuring the likelihood of recovery of an immune cell after a cell membrane damaging event of any of claims 1-4, wherein the level of ADAM-17-cleaved surface receptor expression is assayed within 0 to 24 hours after the immune cell membrane damaging event.

6. The method of measuring the likelihood of recovery of an immune cell after a cell membrane damaging event of any of claims 1-5, wherein the level of ADAM-17-cleaved surface receptor expression is assayed within 12 to 24 hours after the immune cell membrane damaging event.

7. The method of measuring the likelihood of recovery of an immune cell after a cell membrane damaging event of any of claims 1-6, wherein the ADAM-17-cleaved surface receptor expressed on the immune cells comprises CD16, CD62L, or IL-15 receptor (IL-15R).

8. The method of measuring the likelihood of recovery of an immune cell after a cell membrane damaging event of any of claims 1-7, wherein the immune cell comprises a T cell, Natural Killer (NK) cell, chimeric antigen receptor (CAR) T cell, CAR NK cell, macrophage, dendritic cell, natural killer T (NKT) cell, innate lymphoid cell (ILC), B cell, γδT cell, or neutrophil.

9. The method of measuring the likelihood of recovery of an immune cell after a cell membrane damaging event of any of claims 1-8, wherein the immune cell is a Natural Killer (NK) cell or CAR NK cell, and the ADAM-17-cleaved surface receptor is CD16.

10. The method of measuring the likelihood of recovery of an immune cell after a cell membrane damaging event of claim 9, wherein the NK cell is an expanded NK cell.

11. The method of measuring the likelihood of recovery of an immune cell after a cell membrane damaging event of any of claims 1-8, wherein the immune cell is a T cell or CAR T cell and the ADAM-17-cleaved surface receptor comprises CD62L or IL-15R.

12. The method of measuring the likelihood of recovery of an immune cell after a cell membrane damaging event of any of claims 1-11, wherein the level of ADAM-17-cleaved surface receptor expression is assayed using flow cytometry.

13. The method of measuring the likelihood of recovery of an immune cell after a cell membrane damaging event of any of claims 1-12, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

14. A method of administering an immunotherapy to a subject in need thereof, comprising
a) obtaining one or more immune cells previously subjected following a cell membrane damaging event;
b) assaying the immune cells to determine an expression level of an ADAM-17-cleaved surface receptor; and
c) administering to the subject a therapeutically effective amount of immune cells that express an increased level of ADAM-17-cleaved surface receptor relative to a control immune cell.

15. The method of administering an immunotherapy of claim 14, further comprising using the assay results of expression level of an ADAM-17-cleaved surfaced receptor for screening or selecting manufacturing lots of an immune cell therapeutic; and

16. The method of administering an immunotherapy of claim 14 or 15, wherein the immunotherapy is anticancer treatment for a subject who has been diagnosed as having cancer.

17. The method of administering an immunotherapy any of claims 14-16, wherein the cell membrane damaging event comprises a freeze thaw cycle, gene editing, electroporation, magnetofection, detergent permeabilization, strotolysin O (SLO) exposure, physical morphology change (cell squeezing), and/or ethanol exposure.

18. The method of administering an immunotherapy claim 17, wherein the cell membrane damaging event comprises a freeze thaw cycle.

19. The method of administering an immunotherapy of claim 18, wherein immune cells were thawed using a water bath.

20. The method of administering an immunotherapy of any of claims 14-19, wherein the level of ADAM-17-cleaved surface receptor expression is assayed within 0 to 24 hours after the cell membrane damaging event.

21. The method of administering an immunotherapy of any of claims 14-19, wherein the level of ADAM-17-cleaved surface receptor expression is assayed within 12 to 24 hours after the cell membrane damaging event.

22. The method of administering an immunotherapy of any of claims 14-21, wherein the ADAM-17-cleaved surface receptor expressed on the immune cells comprises CD16, CD62L, or IL-15 receptor (IL-15R).

23. The method of administering an immunotherapy of any of claims 14-22, wherein the immune cell comprises a T cell, Natural Killer (NK) cell, chimeric antigen receptor (CAR) T cell, CAR NK cell, macrophage, dendritic cell, natural killer T (NKT) cell, innate lymphoid cell (ILC), B cell, γδT cell, or neutrophil.

24. The method of administering an immunotherapy of any of claims 14-23, wherein the immune cell is a Natural Killer (NK) cell or CAR NK cell, and the ADAM-17-cleaved surface receptor is CD16.

25. The method of administering an immunotherapy of claim 24, wherein the NK cell is an expanded NK cell.

26. The method of administering an immunotherapy of any of claims 14-25, wherein the immune cell is a T cell or CAR T cell and the ADAM-17-cleaved surface receptor comprises CD62L or IL-15R.

27. The method of administering an immunotherapy of any of claims 14-26, wherein the level of ADAM-17-cleaved surface receptor expression is assayed using flow cytometry.

28. The method of administering an immunotherapy of any of claims 14-27, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

29. Use in an immunotherapy of a therapeutically effective amount of immune cells previously subjected following a cell membrane damaging event, wherein the cells express an increased level of ADAM-17-cleaved surface receptor relative to a control immune cell.

30. The use of claim 29, wherein the immunotherapy is an anticancer treatment.

31. The use of claim 29 or 30, wherein the cell membrane damaging event comprises a freeze thaw cycle, gene editing, electroporation, magnetofection, detergent permeabilization, strotolysin O (SLO) exposure, physical morphology change (cell squeezing), and/or ethanol exposure.

32. The use of claim 29 or 30, wherein the cell membrane damaging event comprises a freeze thaw cycle.

33. The use of claim 32, wherein the immune cells were thawed using a water bath.

34. The use of any of claims 29-33, wherein the level of ADAM-17-cleaved surface receptor expression is determined within 0 to 24 hours after the cell membrane damaging event.

35. The use of any of claims 29-33, wherein the level of ADAM-17-cleaved surface receptor expression is assayed within 12 to 24 hours after the cell membrane damaging event.

36. The use of any of claims 29-35, wherein the ADAM-17-cleaved surface receptor expressed on the immune cells comprises CD16, CD62L, or IL-15 receptor (IL-15R).

37. The use of any of claims 29-36, wherein the immune cells comprise T cell, Natural Killer (NK) cell, chimeric antigen receptor (CAR) T cell, CAR NK cell, macrophage, dendritic cell, natural killer T (NKT) cell, innate lymphoid cell (ILC), B cell, γδT cell, or neutrophil.

38. The use of any of claims 29-37, wherein the immune cells comprise Natural Killer (NK) cells and/or CAR NK cells, and the ADAM-17-cleaved surface receptor is CD16.

39. The use of claim 38 wherein the NK cells and/or CAR NK cells comprise expanded NK cells.

40. The use of any of claims 29-36, wherein the immune cells comprise T cells and/or CAR T cells and the ADAM-17-cleaved surface receptor comprises CD62L or IL-15R.

41. The use of any of claims 29-40, wherein the level of ADAM-17-cleaved surface receptor expression is assayed using flow cytometry.

42. The use of any of claims 29-41, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

43. An immunotherapeutic composition comprising a therapeutically effective amount of immune cells previously subjected following a cell membrane damaging event, and expressing an increased level of ADAM-17-cleaved surface receptor relative to a control immune cell.

44. The immunotherapeutic composition of claim 43, wherein the cell membrane damaging event comprises a freeze thaw cycle, gene editing, electroporation, magnetofection, detergent permeabilization, strotolysin O (SLO) exposure, physical morphology change (cell squeezing), and/or ethanol exposure.

45. The immunotherapeutic composition of claim 43 or 44, wherein the membrane damaging event comprises a freeze thaw cycle.

46. The immunotherapeutic composition of claim 45, wherein the immune cells were thawed using a water bath.

47. The immunotherapeutic composition of any of claims 43-46, wherein the level of ADAM-17-cleaved surface receptor expression is determined within 0 to 24 hours after the cell membrane damaging event.

48. The immunotherapeutic composition of any of claims 43-46, wherein the level of ADAM-17-cleaved surface receptor expression is assayed within 12 to 24 hours after the cell membrane damaging event.

49. The immunotherapeutic composition of any of claims 43-48, wherein the ADAM-17-cleaved surface receptor expressed on the immune cells comprises CD16, CD62L, or IL-15 receptor (IL-15R).

50. The immunotherapeutic composition of any of claims 43-49, wherein the immune cells comprise T cell, Natural Killer (NK) cell, chimeric antigen receptor (CAR) T cell, CAR NK cell, macrophage, dendritic cell, natural killer T (NKT) cell, innate lymphoid cell (ILC), B cell, γδT cell, or neutrophil.

51. The immunotherapeutic composition of claim 50, wherein the immune cells comprise Natural Killer (NK) cells and/or CAR NK cells, and the ADAM-17-cleaved surface receptor is CD16.

52. The immunotherapeutic composition of claim 51, wherein the NK cells and/or CAR NK cells comprise expanded NK cells.

53. The immunotherapeutic composition of claim 50, wherein the immune cells comprise T cells and/or CAR T cells and the ADAM-17-cleaved surface receptor comprises CD62L or IL-15R.

54. The immunotherapeutic composition of any of claims 43-53, wherein the level of ADAM-17-cleaved surface receptor expression is assayed using flow cytometry.

55. The immunotherapeutic composition of any of claims 43-54, wherein the level of ADAM-17-cleaved surface receptor expressed on the immune cells is expressed as a ratio of the level of surface receptor cleaved by ADAM17 expressed on the cell membrane damaged immune cell compared with the normal level of ADAM-17-cleaved surface receptor expressed on the immune cells.

56. The immunotherapeutic composition of any of claims 43-55, further comprising a pharmaceutically acceptable carrier.
